(19) United States
(12) Patent Application Publication
(10) Pub. No.: US 2010/0076539 A1
(43) Pub. Date: Mar. 25, 2010

(54) SYSTEM FOR INTRODUCING AN INTRALUMINAL ENDOPROSTHESIS AND METHOD FOR MANUFACTURING SUCH A SYSTEM

(75) Inventors: Bjoern Klocke, Zurich (CH); Matthias Fringes, Ansbach (DE); Claus Martini, Zurich (CH); Stefan Mueller, Zurich (CH)

Correspondence Address: BARNES & THORNBURG LLP, Suite 1150, 3343 Peachtree Road, N.E., Atlanta, GA, 30326-1428, US

(73) Assignee: Biotronik VI Patent AG, Baar (CH)

(21) Appl. No.: 12/371,019

(22) Filed: Feb. 13, 2009

(30) Foreign Application Priority Data
Feb. 13, 2008 (DE) 10 2008 008 926.5

(51) Int. Cl. A61F 2/06; A61M 29/00; A61L 33/00; B29D 23/00; B23P 11/00

(52) U.S. Cl. 623/1.11; 604/103.02; 427/2.24; 264/573; 29/505

(57) ABSTRACT

A system for introducing an intraluminal endoprosthesis (30), preferably a stent, into a body cavity. The system consists of the intraluminal endoprosthesis (30) and a catheter with a balloon (10), the balloon (10) having at least one wing (12) in an undilated condition. The system has at least one active pharmaceutical substance (20) arranged at least partially beneath the at least one wing (12) of the balloon (10) in the undilated condition. The intraluminal endoprosthesis (30) is fixedly arranged on the folded balloon (10) such that the endoprosthesis surrounds the balloon at least partially and protects the at least one active pharmaceutical substance (20) from being washed out and rubbed off. Further disclosed is a method for manufacturing such a system.

### PRIORITY CLAIM

This patent application claims priority to German Patent Application No. 10 2008 008 926.5, filed Feb. 13, 2008, the disclosure of which is incorporated herein by reference in its entirety.

### FIELD

The present disclosure relates to a system for introducing an intraluminal endoprosthesis, preferably a stent, into a body cavity. The system comprises the intraluminal endoprosthesis and a catheter with a balloon, whereby the balloon, in an undilated condition, has at least one wing. The present disclosure also relates to a method for manufacturing such a catheter.

### BACKGROUND

Intraluminal endoprostheses in the form of stents are in wide use at the present time because they allow easy and inexpensive treatment. Intraluminal endoprostheses often have a tubular or hollow cylindrical basic mesh that is open at both longitudinal ends. The basic mesh of such an endoprosthesis is inserted by means of a catheter into the body cavity to be treated and is dilated or released there (for example, Nitinol stents). After removal of the catheter, the endoprosthesis serves to support the body cavity. Such stents have become established for treatment of vascular diseases, in particular. Constricted areas of the blood vessels can be expanded through the use of stents resulting in an increase in lumen.

For purposes of the present disclosure, catheters are tubes or tubing of various diameters that can be inserted into the respective body cavity to be treated. So-called balloon catheters are inserted primarily during angioplasty to widen or re-open a blood vessel. A guide wire is first inserted into the vessel to be treated, and then the balloon catheter, comprising at least one tube having a folded undilated balloon in a predefined area along the tube, is advanced along the guide wire up to the location to be treated in the vessel so that the balloon is positioned in the area of the location in the vessel to be treated, where there is a stenosis, for example. The balloon is then dilated, i.e., unfolded and expanded, so that the location to be treated is reopened and widened, and the flow of body fluid in the vessel is no longer hindered or is not hindered to the previous extent. Finally, the balloon is evacuated and extracted from the vessel along the guide wire. Either at the same time or subsequently, the guide wire is retracted from the vessel.

Such balloon catheters may also be used to introduce intraluminal endoprostheses to a location in a body cavity that is to be treated.

Intraluminal endoprostheses today are often provided with active pharmaceutical substances, such as medicines, which are released in the body over a certain period of time.

Such active pharmaceutical substances may serve to prevent restenoses or agglomerations, for example. Through the release of active pharmaceutical substances that are provided on such intraluminal endoprostheses, it is possible to perform merely a local treatment, i.e., elution of an active ingredient essentially only into the tissue surrounding the intraluminal endoprosthesis. This process is also known as “local drug delivery” (hereinafter, “LDD”). The site of treatment where the active ingredient should manifest its physiological effect thus borders directly at the site of implantation of the intraluminal endoprosthesis.

For purposes of the present disclosure, the term “active pharmaceutical substance” (or active or effective therapeutic substance) means an active ingredient (medication) or a hormone from a plant, animal or synthetic source that is used in a suitable dosage as a therapeutic agent for influencing conditions or functions of the body, as a substitute for natural active ingredients, such as insulin, that are produced by the human or animal body, or for eliminating or rendering harmless not only pathogens that cause diseases but also tumors, cancer cells and exogenous substances. Release of the substance in the vicinity of the endoprosthesis has a positive effect on the course of healing or counteracts pathological changes in the tissue as a result of the surgical procedure and/or serves to render malignant cells harmless in oncology.

Such active pharmaceutical substances have an anti-inflammatory and/or anti-proliferative and/or spasmolytic action, for example, so that restenoses, inflammations or (vascular) spasms can be prevented. In especially preferred exemplary embodiments, such substances may comprise one or more substances from the group consisting of active ingredients including calcium channel blockers, lipid regulators (such as fibrates), immunosuppressants, calcineurin inhibitors (such as tacrolimus), antiphlogistics, and the like (such as cortisone or diclofenac), the anti-inflammatories (such as imidazoles), anti-allergies, oligonucleotides (such as dODN), estrogens (such as genisteine), the endothelium-forming agents (such as fibrin), steroids, proteins, hormones, insulins, cytostatics, peptides, vasodilators (such as sartanes) and the antiproliferative active ingredients, such as taxols or taxans, here preferably paclitaxel or sirolimus.

Currently intraluminal endoprostheses made of a material that is subject to biodegradation are also used. For purposes of the present disclosure, the term “biodegradation” means hydrolytic, enzymatic or other metabolic degradation processes in a living organism that are caused mainly by the body fluids coming in contact with the endoprosthesis and leading to a gradual dissolution of at least large portions of the endoprosthesis. For purposes of the present disclosure, the term “biocorrosion” is often used o as a synonym for the term biodegradation. For purposes of the present disclosure, the term bioabsorption comprises the subsequent absorption of degradation products by the living organism. Such biodegradable materials may be made of polymers or metals. In conjunction with stents, the abbreviation AMS (absorbable metal stent) is also customary. Such stents contain a biodegradable metal, preferably magnesium, iron, zinc, tungsten and/or an alloy of the metals noted hereinabove.

In the case of intraluminal endoprostheses comprising a biodegradable material and also provided with an active pharmaceutical substance, the problem often occurs that the active pharmaceutical substance does not adhere properly to the basic mesh of the endoprosthesis or does not function in the desired manner because, in biodegradation of the endoprosthesis, the pH of the environment may change and/or the endoprosthesis may be corroded in an uncontrollable manner and there may, therefore, be a great deal of infiltration. The active pharmaceutical substance is thus released in an uncontrolled manner and/or not in the desired manner or within the desired period of time.

German Patent Application No. 691 19 753 describes a balloon catheter comprising a catheter body and a balloon which is arranged along the length of the catheter body. The balloon is also provided with a device for inflating and deflating the balloon from the outside and a device for supplying a medication or a combination of medications for treatment or diagnosis within a hollow organ of the body when the catheter is positioned within the hollow organ and inflated. The supply device has microcapsules on the outside of the balloon, such that the microcapsules are secured on the balloon in the folds on the balloon, such as the folds formed when the balloon is deflated. The microcapsules are designed to be rupturable or degradable. The microcapsules open when deposited on the walls of the hollow organ. The capsules may also disintegrate as a result of application of ultrasonic waves.

The disadvantage of the catheter described in German patent Application No. 691 19 753 is that microencapsulated medications, which are expensive to manufacture, are necessary for implementation of the release of the medication. Furthermore, microencapsulation is not feasible for all medications. Also, a balloon of such a catheter provided with microcapsules has a comparatively large diameter; its profile cannot be used in practice and it makes the catheter rigid and inflexible. In addition, the medication may not only be rubbed off at the location that is to be treated but may also be rubbed off during insertion or dilatation of the balloon. This increases the adverse effects associated with the treatment.

### SUMMARY

The present disclosure describes several exemplary embodiments of the present invention.

One aspect of the present disclosure provides a system for introducing an intraluminal endoprosthesis, such as a stent, into a body cavity, the system comprising a) an intraluminal endoprosthesis, and b) a catheter with a balloon, whereby the balloon has an undilated and a dilated condition and has at least one wing in an undilated condition, wherein at least one active pharmaceutical substance is arranged at least partially beneath the at least one wing of the balloon in the undilated condition, and the intraluminal endoprosthesis is attached to the folded balloon so that the intraluminal endoprosthesis at least partially surrounds the balloon.

Another aspect of the present disclosure provides a method for manufacturing a system for introducing an intraluminal endoprosthesis, preferably a stent, into a body cavity, the system comprising an intraluminal endoprosthesis, and a catheter with a balloon, whereby the balloon has at least one wing in an undilated condition, wherein at least one active pharmaceutical substance is arranged at least partially beneath the at least one wing of the balloon in the undilated condition, and the intraluminal endoprosthesis is attached to the folded balloon so that the intraluminal endoprosthesis at least partially surrounds the balloon, the method comprising a) applying at least one active pharmaceutical substance, optionally embedded in a carrier, to the outer surface of the balloon portion of a catheter and an intraluminal endoprosthesis, the active pharmaceutical substance is preferably applied either by immersion, spraying, brush-painting or pressing; b) providing the balloon with at least one wing; c) tightly wrapping the at least one wing; and d) arranging the intraluminal endoprosthesis on the folded balloon such that the endoprosthesis at least partially surrounds the balloon.

One aspect of the present disclosure creates a system that limits the release of an active pharmaceutical substance on introduction of an intraluminal endoprosthesis to the location where the intraluminal endoprosthesis is to be used. Another aspect of the present disclosure provides a method for manufacturing such systems that will be simple and inexpensive.

One aspect defined hereinabove is achieved by a system in which at least one active pharmaceutical substance is arranged at least partially beneath the at least one wing of the balloon in the undilated condition, such that the intraluminal endoprosthesis is securely arranged on the folded balloon of the catheter in such a way that it at least partially surrounds the balloon.

The “undilated condition” here includes all conditions of the balloon of the catheter in which it is not completely unfolded, i.e., at least one wing is at least partially present on the balloon, even if the balloon is already partially expanded. Likewise, conditions in which the balloon is deflated, i.e., has already been dilated once and then collapsed again, are also included here. However, for purposes of the present disclosure, the term “undilated condition” means conditions in which the balloon is present in a completely folded form after folding and impressing and/or is folded up again almost completely after deflation.

Furthermore, for purposes of the present disclosure, the phrase “arranged beneath the at least one wing of the balloon” means that the at least one active pharmaceutical substance is arranged or located within the folds of the balloon, i.e., in or on the surfaces of the wing situated one above the other after folding. The active pharmaceutical substance arranged beneath the at least one wing is thus covered by the portion of the respective wing that is situated on the outside.

In addition, for purposes of the present disclosure, the statement that the endoprosthesis at least partially surrounds the folded balloon means the endoprosthesis is arranged on the outside of the surfaces of the balloon that are on the outside after folding. The endoprosthesis covers these exterior surfaces at least partially. The arrangement of the endoprosthesis and the balloon is not to be regarded as permanent. The endoprosthesis is o arranged fixedly on the balloon until the start of dilatation of the balloon, i.e., even during storage and insertion into the human or animal body. After reaching the maximally dilated condition when the balloon is emptied again, the endoprosthesis remains in the body cavity while the catheter is removed from the body cavity.

Through the system defined above, the active pharmaceutical substance which is not present in microencapsulated form is released to the surrounding body fluid and tissue directly at the site where the intraluminal endoprosthesis is implanted in the body cavity. Before being dispensed, the active pharmaceutical substance is protected from being rubbed off or washed off. Furthermore, the fixed arrangement of the intraluminal endoprosthesis on the balloon ensures that the system of intraluminal endoprosthesis and balloon will assume a very small volume on the whole on introduction of the system into the body cavity, so that the system of intraluminal endoprosthesis and balloon is flexible and easy to handle. Furthermore, it is not necessary to supply the active pharmaceutical substance in a microencapsulation. In addition, the release of the at least one active pharmaceutical substance may occur immediately after dilation of the balloon together with the intraluminal endoprosthesis thus resulting in a rapid onset of effect of the active pharmaceutical substance and an effect precisely at the site of treatment.

In one exemplary embodiment of a catheter of the present disclosure, the intraluminal endoprosthesis is crimped onto the balloon. This is a very simple and inexpensive means of applying the intraluminal endoprosthesis to the balloon.

Furthermore, it is advantageous if the intraluminal endoprosthesis is a biodegradable stent, preferably as an absorbable metal stent. Such a stent is no longer present in the tissue of the treated body cavity after the stent has fulfilled its therapeutic function (the stent dissolves almost completely), and therefore the stent causes fewer adverse effects.

In another exemplary embodiment, the at least one active pharmaceutical substance is arranged in or on one or more vehicles which are arranged beneath a wing or beneath multiple wings of the balloon. It is possible in this way, in an especially simple manner, to reuse a catheter that has already been previously used for introducing an intraluminal endoprosthesis while releasing an active pharmaceutical substance because one or more vehicles can easily be arranged beneath the wings.

It is also especially preferable if the wings of the balloon are adhesively bonded by means of the at least one active pharmaceutical substance. This increases the effect whereby the active pharmaceutical substance does not escape from the folds in an uncontrolled manner and the adhesive pull-away force is increased.

Especially good coverage of the balloon provided with active pharmaceutical substance is then achieved in the undilated condition when, in a preferred exemplary embodiment, the intraluminal endoprosthesis completely covers the underlying outer surfaces of the folded balloon. For purposes of the present disclosure, the phrase “outer surfaces” means the exterior surfaces or surface areas of the folded balloon that are on the outside after folding and impressing. These outer surfaces do not include the outer surfaces or surface areas of the balloon that are arranged beneath the wing or wings and lie one above the other.

The catheter of the system can be manufactured especially inexpensively if the at least one active pharmaceutical substance is applied to the balloon by immersing, spraying, brush-painting or pressing.

In yet another exemplary embodiment, the intraluminal endoprosthesis is provided luminally with a readily dissolvable coating, preferably containing one or more of the substances from the group consisting of (a) sugars, preferably polysaccharides, glycans, glucose, glycogen, amylose, amylopectin, chitin, callose and cellulose, and (b) fats, preferably cholesterol, palm oil, partially hydrogenated soy oils and saturated oils. Such a layer is easily washed off by the body fluid after implantation so that no residues of active ingredient remain on the luminal side of the intraluminal endoprosthesis and, therefore, endothelialization is not hindered.

In another exemplary embodiment, the balloon of the disclosed system comprises at least one folding element running essentially in the longitudinal direction and arranged with a minimum bending radius in one of the areas of the wings. This means that the balloon is folded along the folding element. The correct and reproducible arrangement of the wings on the balloon is achieved by means of such a folding element.

For purposes of the present disclosure, the term “longitudinal direction” means the direction of the axis of the catheter. For purposes of the present disclosure, the phrase “folding element running essentially in the longitudinal element” means a folding element running primarily in the longitudinal direction, i.e., the folding element may also run obliquely or in a spiral in the longitudinal direction, i.e., with one component in a direction perpendicular to the longitudinal direction. Folds or fold lines are areas of the balloon membrane which have a minimum bending radius. These folds occur when the balloon membrane is overstretched on the outside (on the outer wing end) or on the inside (at the point of connection of neighboring wings). For purposes of the present disclosure, the term “folds” in conjunctions with “folding of the balloon” means the initial folding of the balloon in the manufacture of the catheter as well as refolding (also referred to as rewrapping) in deflation.

It is especially preferable if the balloon forms an area of the balloon along the at least one folding element which has an altered thickness, preferably a lower stiffness, in comparison with the other areas of the balloon. In this way, folding along the fold line of the balloon which is predefined by the folding element is facilitated because the balloon membrane always gives at the weakest point as soon as the balloon is deflated.

A preferred and simple possibility for integrating differences in stiffness into the balloon membrane consists of providing recesses or elevations in the balloon or at least one sudden change in wall thickness in the area of the at least one folding element.

Alternatively or in addition to the possibilities mentioned hereinabove for integrating differences in stiffness into the balloon, there is also the advantageous possibility that the at least one folding element forms an area of the balloon having a material composition that is different from that of the other areas of the balloon. These areas (running with the greatest extent in the longitudinal direction of the balloon and having a square, circular, ellipsoidal or rectangular cross section, for example) are designed in the form of a web, for example, where such a web may be provided on the surface of the balloon or embedded in the volume of the balloon. For example, in the case of a balloon material made of PEBAX® (polyether block amide), in the longitudinal direction, areas with a material PA12 running in the longitudinal direction on the outside along the balloon may be introduced into a balloon material on the outside along the balloon, such that these areas remain standing in folding under a greater pull-away force than the balloon material. In folding the balloon, these areas are thus arranged at the minimums in the bending radius, where one wing of the balloon is adjacent to the neighboring wing. Conversely, when using PA12 as a balloon material, areas of the material PEBAX running in the longitudinal direction on the inside along the balloon may also be introduced. These areas are then arranged at the tip of a wing when the balloon is folded because these areas are under a lower tensile stress than the rest of the balloon material. The two exemplary embodiments may also be combined. Additional material combinations may also utilize the materials PA11 and PVC. In another exemplary embodiment in which the materials mentioned hereinabove can also be used, the material of the balloon that does not belong to the folding elements is provided with a reinforcing layer that creates the areas of increased wall thickness. The reinforcing area can be embedded as an intermediate layer into the material of the balloon.

In another exemplary embodiment, the at least one folding element has interruptions that ensure a greater stability of the folding lines.

Also preferred is a system in which the folding element on the balloon runs at a fixed predefined angle to the balloon axis. This also means that the respective wing is created at an angle to the balloon axis. The at least one folding element here runs around the balloon in or on the surface of the balloon and does not merely run parallel to the balloon axis. This creates a uniform bending moment of the folded balloon about its longitudinal axis. With an externally applied bending of the system, the tensile forces and compressions largely cancel one another out, and the strains and stretching compensate for one another. A catheter according to the present disclosure has a uniform trackability because of the angle-independent bending moment of the folded balloon. Furthermore, the peripheral wings no longer stand up in tight curves because the prevailing forces are compensated, as described above, and the stiff wings no longer stand at a right angle to the direction of stress and thus deformation on their flanks can be better dissipated.

In another exemplary embodiment of a catheter of the present disclosure, the folding elements of the balloon are formed by longitudinal struts which form a structure that is arranged on the inside and/or on the outside of the balloon and supports the balloon at defined locations. This structure, which is also referred to below as a Self-X (self-expanding) structure, has the advantage that the structure can be retrofitted. In addition, depending on the choice of the structure material, very high stresses in folding of the balloon can be absorbed with this structure. The longitudinal struts may also run in a spiral. For example, Nitinol or thermoplastics may be used as the structure materials.

In another exemplary embodiment, the active pharmaceutical substance taxols and/or taxans, especially preferably paclitaxel and/or sirolimus, and/or preferably at least one hyperplastic active ingredient having a distribution coefficient between the distribution coefficients of butanol and water of ≧0.5. The hyperplastic active ingredient optionally contained in the active pharmaceutical substance serves to provide locally limited treatment of potentially hyperproliferative tissue. For example, a cytostatic, a corticoid, a prostacycline, an antioxidant, an agent for inhibiting cell proliferation or an immunosuppressant may be used as the antihyperplastic active ingredient.

The present disclosure also provides a method for manufacturing a system as disclosed hereinabove, the method comprising, in one exemplary embodiment, a catheter with a balloon and an intraluminal endoprosthesis, such that first the at least one active pharmaceutical substance is applied to or introduced into the outer surface of the balloon of the catheter, preferably by means of dipping, spraying, brush-painting or pressing. Then the balloon is provided with at least one wing, and this at least one wing is wrapped tightly (i.e., the balloon is wrapped to the smallest possible diameter and is thermally secured in this position). Next the intraluminal endoprosthesis is fixedly arranged on the folded balloon in such a way that the intraluminal endoprosthesis at least partially surrounds the balloon.

The method of manufacturing the system disclosed herein is inexpensive and is easily performed yielding a system that allows the desired local treatment of the body cavity.

The manufacturing method is further simplified if the intraluminal endoprosthesis is arranged on the balloon by means of crimping.

In another exemplary embodiment, the distal and proximal ends of the balloon are left blank when applying the at least one active pharmaceutical substance, preferably by covering them during the application of the substance. This prevents uncontrolled release of the at least one active pharmaceutical substance from the areas of the proximal or distal end of the balloon which might not be completely surrounded by the endoprosthesis.

For the same reason, a method is provided in which the excess of the at least one active pharmaceutical substance is preferably wiped away from the exterior surfaces of the balloon that are on the outside after folding is removed after the folding step. This ensures that the at least one active pharmaceutical substance will be arranged only beneath the wings. To wipe away the active pharmaceutical substance, for example, porous woodpulp paper, a sponge or the like may be used, optionally impregnated with a solvent.

It has also proven to be especially advantageous that the at least one active pharmaceutical substance is cured or polymerized before arrangement of the intraluminal endoprosthesis on the balloon. Curing may also be performed with the help of a polymer or a solvent on the balloon surface. Then the active pharmaceutical substance adheres especially well to the surface of the balloon. The curing or polymerization is implemented, in particular, by UV radiation, beta radiation and/or a thermal treatment.

The at least one active pharmaceutical substance is preferably applied together with a vehicle or carrier (e.g., a polymer or a solvent). In the case when a solvent is used, the solvent evaporates during and/or after application. Expulsion of the solvent with simultaneous hardening of the polymer carrier is also known as curing. Suitable solvents that may be used preferably include the following substances, depending on the active pharmaceutical substance: DMSO, acetone, ether (diethyl ether), methanol, isopropanol, esters, as well as other suitable alcohols. When using polymers or a polymer-like substance as the carrier and curing aid, attention must be paid to the fact that the polymer or polymer-like substance is readily soluble or releases the medication rapidly. From this standpoint, especially suitable substances include hyaloronic acid, P4HB, polyvinylpyrrolidone, liposomes, nanoparticles, silk proteins and cyclodextrins.

In addition to the active pharmaceutical substance and a carrier that is optionally also applied, contrast media (e.g., Ultravist) and/or inorganic salts (e.g., sodium chloride, o sodium carbonate) and/or organic salts (e.g., salts of acetic acid, citric acid, tartaric acid) as well as other solid additives may be applied. These serve to improve the mechanical adhesion to the surface of the balloon and/or to improve the release of the active pharmaceutical substance to the vascular wall and/or to improve the ability of the vascular wall to absorb the active pharmaceutical substance.

When using a carrier for the active pharmaceutical substance containing a solvent to be expelled, the process disclosed herein is preferably modified so that, after application of the carrier with the solvent and active pharmaceutical substance, the balloon is folded and the excess material on the outside after application is removed. Then the balloon is inflated (dilated) and the solvent is expelled, e.g., by a heat treatment. Next the active pharmaceutical substance, optionally containing additional additives in the carrier as described hereinabove, is affixed to the surface of the balloon. Then the balloon may be folded again and the active pharmaceutical substance may be arranged beneath the wings of the balloon, in which case the balloon must be folded at the same locations as in the first folding, preferably by using a self-folding balloon, so that the at least one active pharmaceutical substance is reliably situated beneath the wings of the balloon.

In an advantageous exemplary embodiment of the disclosed process, the applied active pharmaceutical substance is applied to the balloon when the balloon is in the dilated condition to improve the uniformity of the applied active pharmaceutical substance,

As discussed hereinabove, it is advantageous if the intraluminal endoprosthesis is provided luminally on the balloon with a coating that is easy to wash off, preferably with one or more of the substances from the group consisting of sugars, preferably polysaccharides, glycans, glucose, glycogen, amylose, amylopectin, chitin, callose and cellulose, and fats, preferably cholesterol, palm oil, partially hydrogenated soy oils and saturated oils.

The advantages of an arrangement of at least one folding element on the balloon were already explained hereinabove. A balloon provided with a folding element is reproducibly self-folding. An advantageous manufacturing method for the system disclosed hereinabove includes manufacturing the balloon by blow molding before joining the balloon to the other parts of the catheter, whereby the blow mold is provided with a recess or an elevation, e.g., in the form of round or elongated nubs or interrupted grooves at the location where the at least one folding element is to be formed. Alternatively, the balloon may be manufactured by means of injection blow molding such that at least one defined sudden change in wall thickness is created in the area of the at least one folding element. This sudden change in wall thickness can be produced even in the parison, for example, by using a corresponding extrusion die mold or injection die mold. Differences in stiffness of the balloon are created in injection blow molding because of the recesses and elevations in the blow mold as well as the at least one sudden change in wall thickness.

As already explained hereinabove, it is also advantageous to connect the balloon on its inside and/or its outside to a structure which contains longitudinal struts as folding elements and supports the balloon in certain areas. The supporting structure may be made of a highly elastic material which must still be in contact with the inside wall of the balloon even in the maximally dilated condition (at the rated burst pressure). If the internal pressure within the balloon is lowered, then the balloon is kept open only in the area of the supporting structure and the balloon collapses in the unsupported areas between the longitudinal struts. The course of the folds can, therefore, be controlled with a high precision. Such a structure could be made of two spiral Nitinol wires, for example, which are welded to rings in the neck of the balloon.

The at least one folding element may also be created advantageously by thermally treating the balloon locally in the area of the at least one fold line, preferably before application of the at least one active pharmaceutical substance, e.g., by means of a laser and/or by means of a solvent. Furthermore, the balloon may also be provided with another reinforcing material in the area outside of the at least one fold line, likewise preferably before applying the at least one active pharmaceutical substance.

As explained above, it is advantageous if the at least one folding element is provided in such a way that the at least one folding element runs in or on the surface of the balloon at a fixed predefined angle to the balloon axis.

In another exemplary embodiment, the inner shaft and outer shaft of the catheter are rotated before joining them together and/or are displaced so that the balloon is already connected to the inner shaft and the outer shaft before being rotated and/or displaced. The rotated and/or displaced condition in this exemplary embodiment is the normal condition of the instrument. In dilation, the inner shaft and outer shaft store the torsional energy and thus promote spiral refolding of the balloon in deflation.

### BRIEF DESCRIPTION OF THE DRAWINGS

Various aspects of the present disclosure are described hereinbelow with reference to the accompanying figures.

Additional goals, features, advantages and possible applications of the present invention are derived from the following description of exemplary embodiments on the basis of the figures. All the features described and/or illustrated graphically here, either alone or in any combination, constitute the subject matter of the present disclosure, even independently of how they are combined in the individual claims or their reference back to previous claims.

FIG. 1 shows a cross-sectional view of a first exemplary embodiment of a system of the present disclosure;

FIG. 2 shows a cross-sectional view of a second exemplary embodiment of a system of the present disclosure; and

FIG. 3 shows a cross-sectional view of a third exemplary embodiment of a system of the present disclosure.

### DETAILED DESCRIPTION

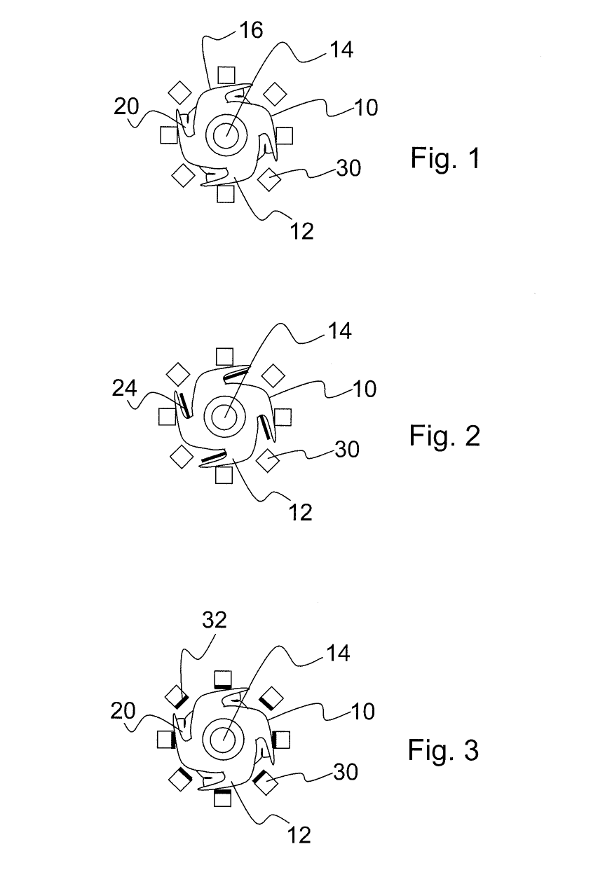

The first exemplary embodiment illustrated in FIG. 1 shows the catheter in the undilated condition. The catheter has a balloon 10 which is arranged in four rings 12, as well as an inner shaft 14 arranged inside the balloon. An active pharmaceutical substance 20 is arranged on the surface of the balloon 10 beneath each of the wings 12. In additional exemplary embodiments of the system of the present disclosure, multiple active pharmaceutical substances may also be provided in the same location.

The intraluminal endoprosthesis in the form of a stent 30 is fixedly arranged with its basic mesh on the surface of the folded balloon 10. In FIG. 1, a slightly widened condition of the system of catheter and stent 30 in comparison with reality is shown (the same thing also applies to FIGS. 2 and 3) to achieve a clearer representation. In comparison with the diagrams in FIGS. 1-3, the wings 12 of the balloon are in close contact with the inner shaft 14, and the stent 30 is in reality crimped tightly onto the balloon 10 of the catheter, so that the outer surfaces 16 of the balloon 10 are almost completely covered by the basic mesh of the stent 30 in the undilated condition. The active pharmaceutical substance is protected by the crimped stent 30 from being washed out/off, e.g., by the bloodstream when the inventive system of the present disclosure is used in a blood vessel.

In a preferred exemplary embodiment, the stent 30 contains a biodegradable material, such as magnesium, a magnesium alloy, iron, tungsten, zinc and/or alloys thereof. By crimping the stent 30, the active pharmaceutical substance is protected by the closed arrangement. Furthermore, on dilation of the balloon, the active pharmaceutical substance is dispensed in a sufficient amount directly to the body cavity into which the catheter is inserted and where the stent 30 has been arranged, such that this peak load has sufficient clinical efficacy. This is the case, in particular, when the balloon 10 returns to the folded condition prior to dilatation by means of folding elements (not shown in the figures) that are provided in the balloon and the active pharmaceutical substance is again protected between the folds.

In the exemplary embodiment illustrated in FIG. 2, the active pharmaceutical substance is contained in or on a lamellar carrier 24. Such a carrier 24 is arranged beneath each wing 12. In this way, in repeated use of the catheter of the system disclosed herein, the catheter is provided with a replenished active pharmaceutical substance or with such a substance coordinated with the corresponding intended purpose and the patients. In additional exemplary embodiments, the active pharmaceutical substance 20 may also be applied by means of a liquid carrier containing a solution or a polymer, for example, as explained hereinabove.

In the exemplary embodiment illustrated in FIG. 3, the stent 30 is additionally provided luminally with a coating 32 which is easily washed off in comparison with the first exemplary embodiment illustrated in FIG. 1. The coating is preferably one or more of the substances from the group consisting of sugars, preferably polysaccharides, glycans, glucose, glycogen, amylose, amylopectin, chitin, callose and cellulose, and fats, preferably cholesterol, palm oil, partially hydrogenated soy oils and saturated oils.

A system as disclosed hereinabove could be manufactured by means of the following processes.

First, the balloon 10 of the catheter is inflated and then coated (by means of immersion or spraying, for example) with one or more active pharmaceutical substances 20 under a slight excess pressure. The distal and proximal ends of the balloon and the balloon cones may be covered so that the distal and proximal ends do not receive any coating of active pharmaceutical substance 20. Next the balloon 10 is folded, i.e., provided with wings, and the wings are brought into contact with the inner shaft 14 to protect the coating. A film may be arranged between the balloon folding device and the balloon itself. In the next step, active pharmaceutical substance 20 which is arranged on the outer surfaces 16 of the wings 12 which form the surface of the folded balloon after folding and arranging the balloon 10, is removed, e.g., by wiping the active pharmaceutical substance away with porous woodpulp paper, a sponge or the like, optionally impregnated with a solvent. Then the active pharmaceutical substance 20 can be cured or polymerized, e.g., by means of UV radiation, beta radiation and/or a thermal treatment. Next the stent 30 is crimped onto the balloon 10 that has been folded and provided with an active pharmaceutical substance 20.

As an alternative to the excess pressure coating with the active pharmaceutical substance, a folded lamellar carrier 24 loaded with a active ingredient may also be introduced into the fold, i.e., beneath the wing 12, and bonded there under the influence of pressure.

In another exemplary embodiment of the manufacturing process, a lamellar carrier with a mixture of the active pharmaceutical substance and a solvent may be clamped beneath a wing 12 and then extracted under the influence of an external pressure so that most of the active pharmaceutical substance is stripped off and remains beneath the wing 12.

All patents, patent applications and publications referred to herein are incorporated by reference in their entirety.

What is claimed is:

1. A system for introducing an intraluminal endoprosthesis, such as a stent, into a body cavity, the system comprising:
a) an intraluminal endoprosthesis, and
b) a catheter with a balloon, whereby the balloon has an undilated and a dilated condition and has at least one wing in an undilated condition, wherein at least one active pharmaceutical substance is arranged at least partially beneath the at least one wing of the balloon in the undilated condition, and the intraluminal endoprosthesis is attached to the folded balloon so that the intraluminal endoprosthesis at least partially surrounds the balloon.

2. The system of claim 1, wherein the intraluminal endoprosthesis is crimped onto the balloon.

3. The system of claim 1, wherein the intraluminal endoprosthesis is a biodegradable stent.

4. The system of claim 1, wherein at least one active pharmaceutical substance is arranged either in or on at least one carrier that are arranged beneath a wing or multiple wings of the balloon.

5. The system of claim 1, wherein the at least one wing of the balloon is adhesively bonded by means of the at least one active pharmaceutical substance.

6. The system of claim 1, wherein the intraluminal endoprosthesis covers the outer areas of the folded balloon which are beneath the endoprosthesis in the undilated condition.

7. The system of claim 1, wherein the at least one active pharmaceutical substance is applied to the balloon by means of either immersion, spraying, brush-painting or pressing.

8. The system of claim 1, wherein the intraluminal endoprosthesis is luminally coated with a coating that is easily dissolvable, preferably with one or more of the substances from the group consisting of (a) one or more sugars selected from the group consisting of polysaccharides, glycans, glucose, glycogen, amylose, amylopectin, chitin, callose, cellulose, and (b) one or more fats selected from the group consisting of cholesterol, palm oil, partially hydrogenated soy oils and saturated oils.

9. The system of claim 1, wherein the balloon provides a folding element that runs essentially in the longitudinal direction which is arranged in an area of the wing running in the longitudinal direction with a minimum bending radius in folding of the balloon.

10. The system of claim 9, wherein the balloon forms an area of the balloon along the at least one folding element such that, in comparison with the other areas of the balloon, said area has a different stiffness, preferably a lower stiffness.

11. The system of claim 10, wherein the balloon has recesses or elevations or at least one sudden change in wall thickness in the area of the at least one folding element.

12. The system of claim 10, wherein the at least one folding element forms an area of the balloon having a material composition different from that of the other areas of the balloon.

13. The system of claim 9, wherein the at least one folding element has interruptions.

14. The system of claim 9, wherein the at least one folding element runs at a fixed predefined angle to the balloon axis.

15. The system of claim 9, wherein the folding elements of the balloon are formed by longitudinal struts which form a structure that is arranged on at least one of the inside and the outside of the balloon and which also supports the balloon in defined locations.

16. The system of claim 9, wherein the at least one active pharmaceutical substance selected from the group consisting of taxols, taxans, paclitaxel, and sirolimus, the at least one active pharmaceutical substance further comprising at least one hyperplastic active ingredient having a distribution coefficient of ≧0.5, between the distribution coefficient of butanol and water.

17. A method for producing a system for introducing an intraluminal endoprosthesis, such as a stent, into a body cavity, the method, comprising:
a) providing a catheter comprising a balloon having a dilated and an undilated state and having at least one wing in the undilated state which has at least one active pharmaceutical substance arranged at least partially beneath the at least one wing of the balloon in the undilated state;
b) providing an intraluminal endoprosthesis attached to a folded balloon so that the intraluminal endoprosthesis at least partially surrounds the balloon;
c) applying at least one active pharmaceutical substance to the outer surface of the balloon by either immersion, spraying, brush-painting or pressing;
d) forming at least one wing on the balloon;
e) bringing the at least one wing into close contact with the balloon; and
f) fixedly associating the intraluminal endoprosthesis on the folded balloon such that the intraluminal endoprosthesis at least partially surrounds the folded balloon.

18. The method of claim 17, wherein the intraluminal endoprosthesis is arranged on the balloon by crimping.

19. The method of claim 17, wherein the balloon has proximal and distal ends, the distal and proximal ends of the balloon remaining substantially uncoated when applying the at least one active pharmaceutical substance.

20. The method of claim 17, further comprising:
e) wiping any excess at least one active pharmaceutical substance from the surfaces of the balloon that are on the outside after folding.

21. The method of claim 17, wherein the at least one active pharmaceutical substance is either cured or polymerized before arranging the intraluminal endoprosthesis on the balloon.

22. The method of claim 17, wherein the at least one active pharmaceutical substance is applied in solution to the balloon by means of a solvent as the carrier, whereby the solvent comprises at least one composition selected from the group consisting of DMSO, acetone, ether (diethyl ether), methanol, isopropanol and esters, before arranging the intraluminal endoprosthesis on the balloon, whereby the solvent is evaporated either during or after application.

23. The method of claim 17, wherein the at least one active pharmaceutical substance is applied and then cured by means of a either polymer or a polymer-like substance as the carrier.

24. The method of claim 17, wherein the active pharmaceutical substance is applied to the balloon when the balloon is in the dilated condition.

25. The method of claim 17, wherein the intraluminal endoprosthesis is luminally coated with a coating that is easily washed off, preferably with one or more of the substances from the group consisting of (a) one or more sugars selected from the group consisting of polysaccharides, glycans, glucose, glycogen, amylose, amylopectin, chitin, callose, cellulose, and (b) one or more fats selected from the group consisting of cholesterol, palm oil, partially hydrogenated soy oils and saturated oils, before arranging the intraluminal endoprosthesis on the balloon.

26. The method of claim 15, wherein at least one folding element is added to the balloon either before or after connecting the balloon to the other parts of the catheter and before the first folding.

27. The method of claim 26, wherein the balloon is manufactured by means of blow molding such that the blow mold is provided with either a recess or an elevation in the location where the at least one folding element is to be formed before joining the balloon to the remaining parts of the catheter.

28. The method of claim 26, wherein the balloon is manufactured by either blow molding or injection blow molding before joining the balloon to the other parts of the catheter such that at least one defined sudden change in wall thickness is created in the area of the at least one folding element.

29. The method of claim 26, wherein the balloon is connected on at least one of the outside and the inside to a structure containing longitudinal struts as folding elements, the structure supporting the balloon in selected areas.

30. The method of claim 26, wherein the balloon is thermally treated locally proximate to the at least one folding element.

31. The method of claim 26, wherein the balloon is treated by means of a solvent in the area of the at least one folding element.

32. The method of claim 26, wherein the balloon is provided with another reinforcing material in the area outside of the at least one folding element.

33. The method of claim 26, wherein the at least one folding element has wings which run at a fixed predefined angle to the balloon axis.

34. The method of claim 17, wherein the inner shaft and the outer shaft of the catheter are either rotated or displaced before joining the inner shaft and the outer shaft of the catheter together, whereby the balloon is already connected to the inner shaft and the outer shaft before being rotated or displaced.
